(19) United States
(12) Patent Application Publication
(10) Pub. No.: US 2007/0135423 A1
(43) Pub. Date: Jun. 14, 2007

(54) E-fluoro-4-(pyridin-2-yl)-piperidine-1-carboxamide derivatives and related compounds which modulate the function of the vanilloid-1 receptor (vr1) for the treatment of pain

(76) Inventors: Tracy Bayliss, Walthamstow (GB); Rebecca Elizabeth Brown, Cambridge (GB); Frank Burkamp, Bishops Stortford (GB); Brian A. Jones, Saffron Walden (GB); Joseph George Neduvelil, London (GB)

Correspondence Address: MERCK AND CO., INC, P O BOX 2000, RAHWAY, NJ, 07065-0907, US

(21) Appl. No.: 10/577,585

(22) PCT Filed: Oct. 27, 2004
(86) PCT No.: PCT/GB04/04538

§ 371 Date: Apr. 27, 2006

(30) Foreign Application Priority Data
Oct. 29, 2003 (GB) 0325287.1

(51) Int. Cl. A61K 31/541; A61K 31/5377; A61K 31/496; A61K 31/4545; A61K 31/454; C07D 417/02; C07D 413/02; C07D 401/02

(52) U.S. Cl. 514/227.5; 544/60; 544/128; 544/360; 546/207; 546/209; 546/192; 546/194; 514/235.2; 514/253.13; 514/316; 514/318; 514/326

(57) ABSTRACT

Compounds of formula (I): wherein: A¹ is phenyl, a six-membered aromatic heterocycle containing one, two or three nitrogen atoms, or a five-membered aromatic heterocycle containing up to four heteroatoms chosen from O, N and S, at most one heteroatom being O or S; A² is phenyl, a six-membered aromatic heterocycle containing one, two or three nitrogen atoms, or a five-membered aromatic heterocycle containing up to four heteroatoms chosen from O, N and S, at most one heteroatom being O or S; L is a bond or C₁₋₆alkylene; R¹ and R² independently chosen from hydrogen and C₁₋₆alkyl or R¹ and R² may, together, form a methylene or ethylene bridge; W is halogen, C₁₋₆alkyl, haloC₁₋₆alkyl, C₁₋₆alkoxy or haloC₁₋₆alkoxy; X is O, S or NR³ or X, together with the atom to which it is attached, and Y, form an unsaturated five-membered ring together with A²; Y is a bond, C₁₋₄alkylene, NH or NH(CH₂)₁₋₃; or a pharmaceutically acceptable salt thereof; other substituents are defined in claim 1; which are useful as therapeutic compounds, particularly in the treatment of pain and other conditions ameliorated by the modulation of the function of the vanilloid-1 receptor (VR1).

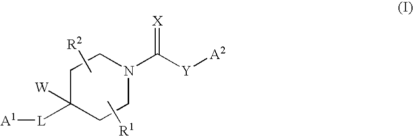

The present invention is concerned with 4-substituted-4-pyridinyl-N-[4-substitutedphenyl]piperidine-1-carboxamides and analogues and derivatives thereof as well as pharmaceutically acceptable salts and prodrugs thereof, which are useful as therapeutic compounds, particularly in the treatment of pain and other conditions ameliorated by the modulation of the function of the vanilloid-1 receptor (VR1).

The pharmacologically active ingredient of chilli peppers has been recognised for some time to be the phenolic amide capsaicin. The application of capsaicin to mucous membranes or when injected intradermally, causes intense burning-like pain in humans. The beneficial effects of topical administration of capsaicin as an analgesic is also well established. However, understanding of the underlying molecular pharmacology mediating these responses to capsaicin has been a more recent development.

The receptor for capsaicin, termed the vanilloid VR1 receptor, was cloned by Caterina and colleagues at UCSF in 1997 (Nature, 398:816, 1997). VR1 receptors are cation channels that are found on sensory nerves that innervate the skin, viscera, peripheral tissues and spinal cord. Activation of VR1 elicits action potentials in sensory fibres that ultimately generate the sensation of pain. Importantly the VR1 receptor is activated not only by capsaicin but also by acidic pH and by noxious heat stimuli. It is also sensitized by a number of inflammatory mediators and thus appears to be a polymodal integrator of painful stimuli.

The prototypical VR1 antagonist is capsazepine (Walpole et al., J Med. Chem., 37:1942, 1994)-VR1 IC₅₀ of 420nM. A novel series of sub-micromolar antagonists has also been reported recently (Lee et al, Bioorg. Med. Chem., 9:1713, 2001), but these reports provide no evidence for in vivo efficacy. A much higher affinity antagonist has been derived from the ‘ultra-potent’ agonist resiniferatoxin. Iodo-resiniferatoxin (Wahl et al., Mol. Pharmacol, 59:9, 2001) is a nanomolar antagonist of VR1 but does not possess properties suitable for an oral pharmaceutical. This last is also true of the micromolar peptoid antagonists described by Garcia-Martinez (Proc. Natl. Acad. Sci., USA, 99:2374, 2002).

WO-A-0208221 (Neurogen Corporation et al.) discloses structurally related VR1 antagonists based around a piperazine core.

We herein describe another novel series of VR1 modulators. These comprise predominantly VR1 antagonists but encompass VR1 partial antagonists and VR1 partial agonists. Such compounds have been shown to be efficacious in animal models of pain.

The present invention provides compounds of formula I: 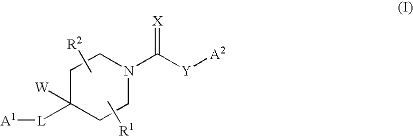
wherein:

A¹ is phenyl, a six-membered aromatic heterocycle containing one, two or three nitrogen atoms, or a five-membered aromatic heterocycle containing up to four heteroatoms chosen from O, N and S, at most one heteroatom being O or S;

A¹ is unsubstituted or substituted by one, two or three substituents independently chosen from halogen, C₁₋₆alkyl, C₂₋₆alkenyl, C₂₋₆alkynyl, haloC₁₋₆alkyl, C₁₋₆alkoxy, haloC₁₋₆alkoxy, hydroxy, cyano, nitro and amino;

A² is phenyl, a six-membered aromatic heterocycle containing one, two or three nitrogen atoms, or a five-membered aromatic heterocycle containing up to four heteroatoms chosen from O, N and S, at most one heteroatom being O or S;

A² is unsubstituted or substituted by one, two or three groups independently chosen from halogen, cyano, nitro, amino, C₁₋₆alkylamino, di(C₁₋₆alkyl)amino, C₁₋₆alkyl C₂₋₆alkenyl, C₂₋₆alkynyl, haloC₁₋₆alkyl, hydroxy, C₁₋₆alkOxy, haloC₁₋₆alkyl, thiol, SF₅, phenylC₁₋₆alkyl and phenyl;

L is a bond or C₁₋₆alkylene;

R¹ and R² independently chosen from hydrogen and C₁₋₆alkyl;

or R¹ and R² may, together, form a methylene or ethylene bridge;

W is halogen, C₁₋₆alkyl, haloC₁₋₆alkyl, C₁₋₆alkoxy or haloC₁₋₆alkoxy;

X is O, S or NR³ where R³ is hydrogen, hydroxy, C₁₋₆alkoxy, C₁₋₆alkyl, cyano, C₃₋₆cycloalkyl, a six-membered saturated heterocycle containing one or two heteroatoms independently chosen from O, N and S, and R³ is, if possible, optionally substituted by C₁₋₆alkyl, C₁₋₆alkoxy, haloC₁₋₆alkyl, haloC₁₋₆alkoxy, halogen, amino, nitro, hydroxy, phenyl, a six-membered aromatic heterocycle containing up to three nitrogen atoms or a five-membered aromatic heterocycle containing up to four heteroatoms chosen from O, N and S, at most one heteroatom being O or S;

or X, together with the atom to which it is attached, and Y, form an unsaturated five-membered ring together with A²;

Y is a bond, C₁₋₄alkylene, NH or NH(CH₂)₁₋₃;

or a pharmaceutically acceptable salt thereof.

A¹ is preferably phenyl or a nitrogen containing heterocycle which is unsubstituted or substituted with one or two groups independently chosen from halogen, hydroxy, cyano, nitro, amino, C₁₋₄alkyl, haloC₁₋₄alkyl, C₁₋₆alkoxy and haloC₁₋₄alkoxy. More particularly A¹ is phenyl, pyridinyl, pyrimidinyl or imidazolyl. Preferred substituents are halogen, C₁₋₄alkyl, C₁₋₄alkoxy and haloC₁₋₄alkyl, such as fluorine, chlorine, methyl, methoxy and trifluoromethyl.

Particular embodiments of A¹ include 3-methylpyrid-2-yl, pyrid-2-yl, 1-methylimidazol-2-yl, 3-chloropyrid-2-yl, 3-fluoropyrid-2-yl, 3-methoxypyrid-2-yl, phenyl, pyrimidin-2-yl and 3-trifluoromethylpyrid-2-yl. Pyridyl, particularly pyrid-2-yl, especially substituted at the 3-position, preferably by methyl, is preferred.

A² is preferably a six-membered aromatic or heteroaromatic ring. A² is preferably monosubstituted, particularly para to the point of attachment to Y.

The substituent is preferably haloC₁₋₆alkyl, C₁₋₆alkyl, SF₅, phenyl, phenylC₁₋₆alkyl, haloC₁₋₆alkoxy, cyano or di(C₁₋₆alkyl)amino. More preferably the substituent is haloC₁₋₄alkyl, C₁₋₄alkyl, SF₅, phenyl, phenylC₁₋₂alkyl, haloC₁₋₄alkoxy, cyano or di(C₁₋₄alkyl)amino. Particular substituents include trifluoromethyl, isopropyl, 1,2,2,2-tetrafluoro-1-trifluoromethylethyl, tert-butyl, SF₅, n-butyl, benzyl, phenyl, 2,2,2-trifluoroethyl, trifluoromethoxy, cyano and dimethylamino.

A² is preferably phenyl or pyridyl, particularly phenyl.

Particular embodiments of A² include 4-trifluoromethylphenyl, 4-isopropylphenyl, 4-(1,2,2,2-tetrafluoro-1-trifluoromethylethyl)phenyl, 4-benzylphenyl, 4-(pentafluoro-λ⁶-sulfonyl)phenyl, biphenyl, 3-trifluoromethylpyrid-6-yl, 4-(2,2,2-trifluoroethyl)phenyl, 4-trifluoromethoxyphenyl, 4-cyanophenyl and 4-dimethylaminophenyL One embodiment is 4-trifluoromethylphenyl.

L is preferably a bond or C₁₋₃alkylene, such as ethylene. Most preferably L is a bond.

R¹ and R² are preferably independently hydrogen, C₁₋₂alkyl or together form a methylene or ethylene bridge. More preferably they are hydrogen, methyl or an ethylene bridge. In one embodiment both are hydrogen.

W is preferably halogen, C₁₋₄alkyl, haloC₁₋₄alkyl, C₁₋₄alkoxy or haloC₁₋₄alkoxy. More preferably W is halogen, C₁₋₂alkoxy or haloC₁₋₂alkyl. Particular embodiments of W include fluorine, methoxy and fluoromethyl.

X is preferably O, S, or NR³ where R³ is hydrogen, hydroxy, C₁₋₆alkyl, C₁₋₆alkoxy, C₃₋₆cycloalkyl, piperidinyl, piperazinyl or morpholinyl optionally substituted, if possible, by C₁₋₆alkyl, C₁₋₆alkoxy, halogen, haloC₁₋₆alkyl, haloC₁₋₆alkoxy, phenyl or pyridyl. More preferably X is O, S or NR³ where R³ is cyano or 1-phenylpiperidin-4-yl. X may be O.

Y is preferably a bond, NH, NHCH₂, CH₂ or CH₂CH₂.

X, the atom to which X is attached, Y and A² may together form an imidazole.

Particular embodiments of the invention include:
4-fluoro-4-(3-methylpyridin-2-yl)-N-[4-trifluoromethylphenyl]piperidine-1-carboxamide;
4-fluoro-4(pyridin-2-yl)N-[4-trifluoromethylphenyl]piperidine-1-carboxamide;
4-fluoro-4(pyridine-2-yl)N-[4-trifluoromethylbenzyl]piperidine-1-carboxamide;
2-{4-fluoro-1-[4-trifluoromethylbenzoyl]piperidin-4-yl}pyridine;
2-(4-fluoro-1-{[4-trifluoromethylphenyl]acetyl}piperidin-4-yl)pyridine;
2-(4-fluoro-1-{3-[4-trifluoromethylphenyl]propanoyl}piperidin-4-yl)pyridine 4-fluoro-4-(1-methyl-1H-imidazol-2-yl)-N-[4-trifluoromethylphenyl]piperidine-1-carboxamide;
4-methoxy-4-pyridin-2-yl-N-[4-trifluoromethylphenyl]piperidine-1-carboxamide;
4-methoxy-4-pyridin-2-yl-N-[4-trifluoromethylbenzyl]piperidine-1-carboxamide;
4-fluoro-N-(4-isopropylphenyl)-4-(3-methylpyridin-2-yl)piperidine-1-carboxamide;
4-fluoro-4-(3-methylpyridin-2-yl)-N-{4-[1,2,2,2-tetrafluoro-1-trifluoromethylethyl] phenyl}piperidine-1-carboxamide;
N-(4-Tert-butylphenyl)-4-fluoro-4-(3-methylpyridin-2-yl)piperidine-1-carboxamide;
4-fluoro-4-(3-methylpyridin-2-yl)-N-[4-(pentafluoro-λ⁶-sulfanyl)phenyl]piperidine-1-carboxamide;
N-(4-Butylphenyl)-4-fluoro-4-(3-methylpyridin-2-yl)piperidine-1-carboxamide;
N-(4-Benzylphenyl)-4-fluoro-4-(3-methylpyridin-2-yl)piperidine-1-carboxamide;
N-biphenyl-4-yl-4-fluoro-4-(3-methylpyridin-2-yl)piperidine-1-carboxamide;
4-fluoro-4-(3-methylpyridin-2-yl)-N-[5-trifluoromethylpyridin-2-yl]piperidine-1-carboxamide;
4-(3-chloropyridin-2-yl)-4-fluoro-N-[4-trifluoromethylphenyl]piperidine-1-carboxamide
4-fluoro-4-(3-fluoropyridin-2-yl)-N-[4-trifluoromethylphenyl]piperidine-1-carboxamide;
4-fluoro-4-(3-methoxypyridin-2-yl)-N-[4-trifluoromethylphenyl]piperidine-1-carboxamide;
4-fluoro-4-(3-methylpyridin-2-yl)-N-[4-trifluoromethylphenyl]piperidine-1-carbothioamide;
N-cyano-4-fluoro-4-(3-methylpyridin-2-yl)-N-[4-trifluoromethylphenyl]piperidine-1-carboximidamide;
4-fluoro-4-(3-methylpyridin-2-yl)-N-(1-phenylpiperidin-4-yl)-N-[4-trifluoromethylphenyl]piperidine-1-carboximidamide;
4-fluoro-4-phenyl-N-[4-trifluoromethylphenyl]piperidine-1-carboxamide;
(+/−)-(syn)-4-fluoro-2-methyl-4-(3-methylpyridin-2-yl)-N-[4-trifluoromethylphenyl]piperidine-1-carboxamide;
4-(fluoromethyl)-4-pyridin-2-yl-N-[4-trifluoromethylphenyl]piperidine-1-carboxamide;
syn- and anti-3-fluoro-3-pyridin-2-yl-N-[4-trifluoromethylphenyl]-8-azabicyclo [3.2.1]octane-8-carboxamide & 3-fluoro-3-pyridin-2-yl-N-[4-trifluoromethylphenyl]-8-azabicyclo[3.2.1]octane-8-carboxamide;
4-fluoro-4-pyrimidin-2-yl-N-[4-trifluoromethylphenyl]piperidine-1-carboxamide;
4-fluoro-4-(3-phenylpropyl)-N-[4-trifluoromethylphenyl]piperidine-1-carboxamiide;
2-[4-fluoro-4-(3-methylpyridin-2-yl)piperidin-1-yl]-6-trifluoromethyl-1H-benzimidazole;
2-(4-fluoro-4-pyridin-2-ylpiperidin-1-yl)-6-(trifluoromethyl)-1H-benzimidazole;
4-fluoro-N-[4-trifluoromethylphenyl]-4-[3-trifluoromethylpyridin-2-yl]piperidine-1-carboxamide;
4-fluoro-N-(4-methylphenyl-4-(3-methylpyridin-2-yl)piperidine-1-carboxamide;
N-(4-ethylphenyl)-4-fluoro-4-(3-methylpyridin-2-yl)piperidine-1-carboxamide;
N-(4-chlorophenyl)-4-fluoro-4-(3-methylpyridin-2-yl)piperidine-1-carboxamide;
4-fluoro-4-(3-methylpyridin-2-yl)-N-[4-trifluoromethoxyphenyl]piperidine-1-carboxamide;
N-(4-cyanophenyl)-4-fluoro-4-(3-methylpyridin-2-yl)piperidine-1-carboxamide;
N-[4-dimethylaminophenyl]-4-fluoro-4-(3-methylpyridin-2-yl)piperidine-1-carboxamide;
or a pharmaceutically acceptable salt thereof.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy. “Alkylthio” shall be construed in an analogous manner.

As used herein, the terms “haloC₁₋₆alkyl” and “haloC₁₋₆alkoxy” means a C₁₋₆alkyl or C₁₋₆alkoxy group in which one or more (in particular, 1 to 3) hydrogen atoms have been replaced by halogen atoms, especially fluorine or chlorine atoms. Preferred are fluoroC₁₋₆alkyl and fluoroC₁₋₆alkoxy groups, in particular, fluoroC₁₋₃alkyl and fluoroC₁₋₃alkoxy groups, for example, CF₃, CH₂CH₂F, CH₂CHF₂, CH₂CF₃, OCF₃, OCH₂CH₂F, OCH₂CHF₂ or OCH₂CF₃, and most especially CF₃ and OCF₃.

As used herein, the terms “alkenyl” and “alkynyl” as a group or part of a group means that the group is straight or branched. Examples of suitable alkenyl groups include vinyl and allyl. A suitable alkynyl group is acetylene or propargyl.

When used herein, the term “halogen” means fluorine, chlorine, bromine and iodine. The most preferred halogens are fluorine and chlorine, especially fluorine.

Examples of 6-memnbered heterocycles are pyridine, pyrimidine, pyrazine, pyridazine and triazine.

Examples of 5-membered heterocycles are thiophene, furan, pyrrole, imidazole, pyrazole, oxazole, isoxazole, thiazole, isothiazole, 1,2,3-triazole, 1,2,4-triazole, oxadiazole, thiadiazole and tetrazole.

In a further aspect of the present invention, the compounds of formula I may be prepared in the form of a pharmaceutically acceptable salt, especially an acid addition salt.

For use in medicine, the salts of the compounds of formula I will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulphonic acid, maleic acid, succinic acid, acetic acid, citric acid, tartaric acid, carbonic acid, phosphoric acid or sulphuric acid. A further salt is the acid addition salt with benzenesulfonic acid. Preferred pharmaceutically acceptable salts of the compounds of the present invention are the besylate salts. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the compound of formula I with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention also includes within its scope N-oxides of the compounds of formula I above. In general, such N-oxides may be formed on any available nitrogen atom. The N-oxides may be formed by conventional means, such as reacting the compound of formula I with oxone in the presence of wet alumina.

The present invention includes within its scope prodrugs of the compounds of formula I above. In general, such prodrugs will be functional derivatives of the compounds of formula I which are readily convertible in vivo into the required compound of formula I. Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The present invention includes within its scope solvates of the compounds of formula I and salts thereof, for example, hydrates.

The compounds according to the invention may have one or more asymmetric centres, and may accordingly exist both as enantiomers and as diastereoisomers. It is to be understood that all such isomers and mixtures thereof are encompassed within the scope of the present invention. Furthermore, the compounds of formula I may also exist in tautomeric forms and the invention includes within its scope both mixtures and separate individual tautomers.

The compounds may exist in different isomeric forms such as syn- and anti-isomers, all of which are encompassed by the present invention.

The present invention further provides pharmaceutical compositions comprising one or more compounds of formula I in association with a pharmaceutically acceptable carrier or excipient.

Preferably the compositions according to the invention are in unit dosage forms such as tablets, pills, capsules, powders, granules, sterile parenteral solutions or suspensions, metered aerosol or liquid sprays, drops, ampoules, auto-injector devices, suppositories, creams or gels; for oral, parenteral, intrathecal, intranasal, sublingual, rectal or topical administration, or for administration by inhalation or insufflation. Oral compositions such as tablets, pills, capsules or wafers are particularly preferred. For preparing solid compositions such as tablets, the principal active ingredient is mixed with a pharmaceutical carrier, e.g. conventional tabletting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation composition is then subdivided into unit dosage forms of the type described above containing from 0.1 to about 500 mg of the active ingredient of the present invention. Favoured unit dosage forms contain from 1 to 500 mg, for example 1, 5, 10, 25, 50, 100, 300 or 500 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of painful conditions such as those listed below, a suitable dosage level is about 1.0 mg to 15 g per day, preferably about 5.0 mg to 1 g per day, more preferably about 5 mg to 500 mg per day, especially 10 mg to 100 mg per day. The compounds may be administered on a regimen of 1 to 4 times per day.

It will be appreciated that the amount of a compound of formula I required for use in any treatment will vary not only with the particular compounds or composition selected but also with the route of administration, the nature of the condition being treated, and the age and condition of the patient, and will ultimately be at the discretion of the attendant physician.

The invention further provides a compound of formula I as defined above, or a pharmaceutically acceptable salt thereof, for use in treatment of the human or animal body. Preferably, said treatment is for a condition which is susceptible to treatment by modulation (preferably antagonism) of VR1 receptors.

The compounds of the present invention will be of use in the prevention or treatment of diseases and conditions in which pain and/or inflammation predominates, including chronic and acute pain conditions. Such conditions include rheumatoid arthritis; osteoarthritis; post-surgical pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labour pain, chronic pelvic pain, chronic prostatitis and endometriosis; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; itching conditions including pruritis, itch due to hemodialysis, and contact dermatitis; pain (as well as broncho-constriction and inflammation) due to exposure (e.g. via ingestion, inhalation, or eye contact) of mucous membranes to capsaicin and related irritants such as tear gas, hot peppers or pepper spray; neuropathic pain conditions such as diabetic neuropathy, chemotherapy-induced neuropathy and post-herpetic neuralgia; “non-painful” neuropathies; complex regional pain syndromes; pain associated with carcinoma, often referred to as cancer pain; central nervous system pain, such as pain due to spinal cord or brain stem damage, low back pain, sciatica and ankylosing spondylitis; gout; scar pain; irritable bowel syndrome; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis, asthma and rhinitis, including allergic rhinitis such as seasonal and perennial rhinitis, and non-allergic rhinitis and cough; autoimmune diseases; and immunodeficiency disorders. The compounds of the present invention may also be used to treat depression. They may also be used to treat gastro-oesophageal reflux disease (GERD), particularly the pain associated with GERD.

Thus, according to a further aspect, the present invention provides a compound of formula I for use in the manufacture of a medicament for the treatment or prevention of physiological disorders that may be ameliorated by modulating VR1 activity.

The present invention also provides a method for the treatment or prevention of physiological disorders that may be ameliorated by modulating VR1 activity, which method comprises administration to a patient in need thereof of an effective amount of a compound of formula I or a composition comprising a compound of formula I.

According to a further or alternative aspect, the present invention provides a compound of formula I for use in the manufacture of a medicament for the treatment or prevention of a disease or condition in which pain and/or inflammation predominates.

The present invention also provides a method for the treatment or prevention of a disease or condition in which pain and/or inflammation predominates, which method comprises administration to a patient in need thereof of an effective amount of a compound of formula I or a composition comprising a compound of formula I.

According to a further aspect of the present invention, it may be desirable to treat any of the aforementioned conditions with a combination of a compound according to the present invention and one or more other pharmacologically active agents suitable for the treatment of the specific condition. The compound of formula I and the other pharmacologically active agent(s) may be administered to a patient simultaneously, sequentially or in combination. Thus, for example, for the treatment or prevention of pain and/or inflammation, a compound of the present invention may be used in conjunction with other analgesics, such as acetaminophen (paracetamol), aspirin and other NSAIDs, including selective cyclooxygenase-2 (COX-2) inhibitors, as well as opioid analgesics, especially morphine, NR2B antagonists, bradykinin antagonists, anti-migraine agents, anticonvulsants such as oxcarbazepine and carbamazepine, antidepressants (such as TCAs, SSRIs, SNRIs, substance P antagonists, etc.), spinal blocks, gabapentin, pregabalin and asthma treatments (such as θ₂-adrenergic receptor agonists or leukotriene D₄ antagonists (e.g. montelukast).

Specific anti-inflammatory agents include diclofenac, ibuprofen, indomethacin, nabumetone, ketoprofen, naproxen, piroxicam and sulindac, etodolac, meloxicam, rofecoxib, celecoxib, etoricoxib, parecoxib, valdecoxib and tilicoxib. Suitable opioid analgesics of use in conjunction with a compound of the present invention include morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine; or a pharmaceutically acceptable salt thereof. Suitable anti-migraine agents of use in conjunction with a compound of the present invention include CGRP-antagonists, ergotamines or 5-HT₁ agonists, especially sumatriptan, naratriptan, zolmatriptan or rizatriptan.

Therefore, in a further aspect of the present invention, there is provided a pharmaceutical composition comprising a compound of the present invention and an analgesic, together with at least one pharmaceutically acceptable carrier or excipient.

In a further or alternative aspect of the present invention, there is provided a product comprising a compound of the present invention and an analgesic as a combined preparation for simultaneous, separate or sequential use in the treatment or prevention of a disease or condition in which pain and/or inflammation predominates.

The compounds of formula I in which Y is NH or NH(CH₂)₁₋₃ can be made by reacting a compound of formula II with a compound of formula III: 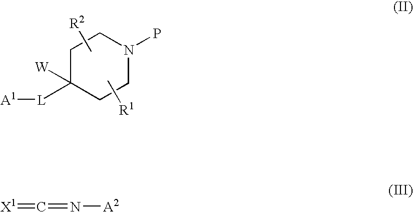
wherein X¹ is O or S, P is H or a C₁₋₆alkoxycarbonyl group such as tert-butoxycarbonyl and A¹, A², L, R¹, R² and W are as defined above. The reaction is generally carried out in a solvent such as dichloromethane in the presence of a base such as triethylamine at about room temperature for about one hour.

The resulting product can be converted into a compound of formula I in which X is NR³ by reacting with a compound of formula VIII:
R³NH₂ (VIII)
in which R³ is as defined above, in the presence of a catalyst such as silver acetate in a solvent such as acetonitrile at about 140° C. for about 10 minutes in a microwave or for several hours at reflux.

The compound of formula II in which W is OH can be made by reacting a compound of formula VI with a compound of formula VII: 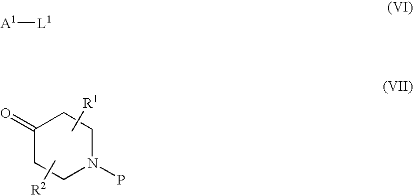
wherein A¹, R¹, R² and P are as defined above and L¹ is a leaving group such as H or Br. The compound of formula VI is first converted to its anion by reacting with a strong base such as N-butyl lithium in hexanes and then reacted with the compound of formula VII generally in a solvent such as THF between −78° C. and room temperature for several hours.

The resulting group W can be converted into other groups W by standard methods known in the art. For example a hydroxy group can be converted to a fluorine atom by reacting with diethylaminosulphur trifluoride in a solvent such as dichloromethane at a temperature between −78° C. and room temperature for about two hours. A hydroxy group can be converted to an alkoxy group by reacting first with a strong base such as sodium hydride in a solvent such as a mixture of tetrahydrofuran and dimethyl formamide for about two hours followed by addition of the appropriate alkyl iodide and leaving to react for about 3 days.

Compounds of formula II in which W is CN can also be made by reacting a compound of formula X with a compound of formula XI:
A¹—CH₂—CN (X)
(ClCH₂CH₂)₂NP (XI)
wherein A¹ and R are as defined above in the presence of a strong base such as sodium hydride in a solvent such as anhydrous dimethylformamide at about 60° C. for about 5½ hours.

The cyano group can be converted to an ester using hydrogen chloride gas in anhydrous methanol. This can be reduced to a hydroxymethyl group using a reagent such as lithium aluminium hydride in a solvent such as tetrahydrofuran at about −30° C. for about one hour. This group can be converted to a fluoromethyl group using a reagent such as diethylaminosulphur trifluoride in a solvent such as anhydrous ethyl acetate at between −78° C. and room temperature for several hours.

Compounds of formula III in which X¹ is O can be made by reacting the corresponding amine with triphosgene followed by a base such as triethylamine in a solvent such as dichloromethane for about one hour.

Compounds of formula m in which X¹ is O can be made by reacting the corresponding carboxylic acid with an azide such as diphenyl phosphoryl azide in the presence of a base such as triethylamine at about 90° C. for about 3 hours so that a Curtius rearrangement occurs.

Compounds of formula VII can be made by hydrogenating a compound of formula IX: 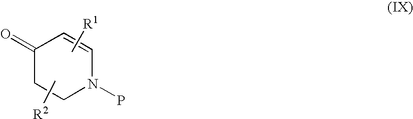
wherein R¹, R² and P are as defined above by stirring with a reducing agent such as zinc powder in a solvent such as acetic acid at reflux for about 18 hours.

In an alternative process, compounds of formula I in which Y is a bond or C₁₋₄alkylene can be made by reacting a compound of formula II with a compound of formula IV: 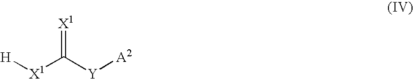
wherein both X¹s are O or S, Y is a bond or C₁₋₄alkylene and A² is as defined above. The reaction is generally carried out in a solvent such as tetrahydrofuran at about 70° C. for several hours. The compound of formula IV may be pre-reacted with an activating agent such as carbonyldiimidazole in a solvent such as tetrahydrofuran for about 3 hours at about 70° C.

In a further process, compounds of formula I in which X, together with the atom to which it is attached, and Y, form an unsaturated five membered ring together with A² can be made by reacting a compound of formula II with a compound of formula V: 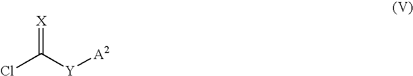
wherein X, together with the atom to which it is attached and Y, form an unsaturated five membered ring together with A². The reaction is generally carried out in a solvent such as ethanol with heating to about 160° C. for about 10 minutes preferably in a microwave.

Where the synthesis of intermediates and starting materials is not described these compounds are commercially available or can be made from commercially available compounds by standard methods.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P. G. M. Wuts, Protective Groups in Organic Synthesis, John Wiley & Sons, 1991. The protecting groups may be removed at a convenient subsequent stage using methods known from the art.

The following Examples serve to illustrate the preparation of compounds of the present invention.

### COMMON INTERMEDIATES

### Description Example 1

### Tert-butyl 4-hydroxy-4-(3-methylpyridin-2-yl)piperidine-1-carboxylate

N-butyl lithium (15.63 ml, 1.6 M in hexanes) was added dropwise to a stirred solution of 2-bromo-3-methyl pyridine (4.3 g, 25 mmol) in THF (35 ml) at −78° C. over 40 minutes. The anion formed went from orange to brown. After 30 minutes at −78° C., 1-tertbutoxycarbonyl-4-piperidone (5 g) in THF (30 ml) was added dropwise over 20 minutes. The reaction mixture was allowed to warm to room temperature overnight. Water (50 ml) was added and the organic layer was separated. The aqueous was extracted using ethyl acetate (2×50 ml). The organics were combined, dried (Na₂SO₄) and evaporated. Purification by column chromatography on silica using 5-10% ethyl acetate in isohexane yielded the desired product (1.72 g).

1H NMR δ(ppm) 400 MHz (CDCl3): 1.44 (2H, d, J=12.9 Hz), 1.50 (9H, s), 2.27-2.34 (2H, m), 2.50 (3H, s), 3.26-3.4 (2H, br m), 4.0-4.2 (2H, br m), 6.63 (1H, s), 7.18 (1H, dd, J=4.7, 7.8 Hz), 7.50(1H, dd, J=1.2, 7.8 Hz), 8.38 (1H, dd, J=1.2, 4.7 Hz). MSp m/z for MH+=293.

### Description Example 2

### Tert-butyl 4-fluoro-4-(3-methylpyridin-2-yl)piperidine-1-carboxylate

Description Example 1 (4.47 g, 15 mmol) in dichloromethane (150 ml) was cooled to −78° C. and excess diethylaminosulphur trifluoride (11.15 g, 69 mmol) in dichloromethane (50 ml) was added dropwise to the stirred solution: The reaction was warmed to room temperature over 2 hours. Water (50 ml) was added and the organic layer separated. The aqueous was extracted with dichloromethane (2×50 ml). The organics were combined, dried (Na₂SO₄) and evaporated. Purification by column chromatography on silica using 10-100% ethyl acetate in isohexane yielded the desired product (2.65 g).

1H NMR 8 (ppm) 400 MHz(CDCl3): 1.48 (9H, s), 1.95-2.1 (2H, br m) 2.15-2.45 (2H, br m) 2.50 (3H, d, J=5.9 Hz), 3.15-3.3 (2H, br m), 4.0-4.2 (2H, br m), 7.12 (1H, dd, J=4.7, 7.8 Hz), 7.46 (1H, dd, J=1.2, 6.7 Hz), 8.35 (1H, d, J=4.7 Hz). MSp m/z for MH+=295.

### Description Example 3

### 2-(4-Fluorolpiperidin-4-yl)-3-methylpyridine

Description Example 2 (1.44 g, 4.9 mmol) in dichloromethane (20 ml) was treated with trifluoroacetic acid (5 ml) and stirred at room temperature for 3 hours. The reaction mixture was evaporated and purified using a strong cation exchange cartridge to give the desired product (710 mg).

1H NMR δ (ppm) 400 MHz(CDCl3): 2.01-2.08 (2H, m), 2.17-2.35 (2H, m), 2.50 (3 H, d, J=5.5 Hz), 3.04-3.11 (4H, m), 7.11 (1H, dd, J=4.7, 7.4 Hz), 7.45 (1H, dd, J=7.4, 0.8 Hz), 8.37 (1H, d, J=4.7 Hz). MSp m/z for MH+=195.

### Description Example 4

### 2-(4-Fluoropiperidin-4-yl)pyridine

The title compound was prepared in analogous fashion to Description Example 3.

1H NMR δ (ppm) 400 MHz (CDCl3): 1.84-1.91 (2H, m), 2.14-2.22 (1H, m), 2.24-2.32 (1H, m), 3.04-3.10(4H m), 7.18-7.22 (1H, m), 7.56 (1H, dd, J=1.2, 7.8Hz), 7.70-7.74 (1H, m), 8.56 (1H, m). MSp m/z for MH+=181.

### Description Example 5

### Tert-butyl 4-hydroxy-4-(1-methyl-1H-imidazol-2-yl)piperidine-1-carboxylate

N-butyl lithium (6.25 ml, 1.6 M in hexanes) was added dropwise to a solution of 1-methylnimdazole in THF (15 ml) at −78° C. After 30 minutes at this temperature, 1-tertbutoxycarbonyl-4-piperidone (2 g) in THF (15 m) was added dropwise. The reaction mixture was allowed to warm to room temperature overnight. Water (50 ml) was added and the organics were separated. The aqueous was extracted using ethyl acetate (2×50 ml). The organics were combined, dried (Na₂SO₄) and evaporated. Purification by column chromatography on silica using 5% methanol in dichloromethane as the eluant gave the desired product (2 g).

1H NMR δ (ppm) 400 MHz (CDCl3): 1.46 (9H, s), 1.77-1.85 (2H, m), 2.05-2.18 (2 H, br m), 3.18 (1H, br s), 3.26-3.37 (2H, br m), 3.8-3.93 (2H, br m), 3.84 (3H, s), 6.81 (2H, s). MSP m/z for MH+=282.

### Description Example 6

### Tert-butyl 4-methoxy-4-(pyridin-2-yl)piperidine-1-carboxylate

Description Example 1 (557 mg, 2 mmol) in THF (5 ml) was treated with sodium hydride (56 mg, 95% dry, 2.2 mmol) and stirred at room temperature for 20 minutes. Dimethylformamide (5 ml) was added to aid solubility of the anion formed. Iodomethane (131 μl) was added. After 2 hours and 4 hours, further iodomethane (50 μl) was added. The reaction was stirred for 72 hours. Water (20 ml) was added and the aqueous was extracted using ethyl acetate (3×20 ml). The combined organics were dried (Na₂SO₄), filtered and evaporated. The compound was purified by column chromatography using 20% ethyl acetate in isohexane as eluant to give the desired material (250 mg). 1H NMR δ (ppm) 400 MHz (CDCl3): 1.46 (9H, s), 1.95-1.99 (2H, m), 2.05-2.12 (2H, m), 3.06 (3H, s), 3.18-3.24 (2H, m), 3.92-3.95 (2H, m), 7.20 (1H, dd, J=1.2, 12.1 Hz), 7.48 -7.50 (1H, m), 7.69-7.73 (1H, m), 8.57-8.59 (1H, m).

### FINAL PRODUCTS

### Example 1

### 4-Fluoro-4-(3-methylpyridin-2-yl)-N-[4-trifluoromethylphenyl]piperidine-1-carboxamide

Trifluoroacetic acid (2 ml) was added to a stirred solution of Description Example 2 (510 mg, 1.73 mmol) in dichloromethane (8 ml). After 1 hour, the reaction was complete and the solvent was removed by evaporation. The residue was dissolved in dichloromethane (10 ml), treated with triethylamine (1.25 ml) followed by 4-trifluoromethylphenyl isocyanate (324 mg, 1.73 mmol) and stirred at room temperature for 1 hour. The reaction mixture was evaporated and purified by column chromatography on silica using 20-33% ethyl acetate in isohexane to give the desired product (470 mg).

1H NMR δ (ppm) (CDCl3): 2.13-2.20 (2H, m), 2.37-2.49 (2H, m), 2.52 (3 H, d, J=5.9 Hz), 3.38-3.48 (2H, m), 4.04-4.11 (2H, m), 6.57 (1H, s), 7.15 (1 H, dd, J=4.7, 7.8 Hz), 7.48-7.56 (5H, m), 8.35 (1H, d, J=4.7 Hz). MSp m/z for MH+=382.

### Example 2

### 4-Fluoro-4(pyridin-2-yl)N-[4-trifluoromethylphenyl]piperidine-1-carboxamide

Using Description Example 4 and the procedure shown in Example 1, the title compound was obtained.

1H NMR δ (ppm) 400 MHz(CDCl3): 1.96-2.04 (2H, m), 2.32-2.50 (2H, m), 3.37-3.44 (2H, m), 4.09-4.13 (2H, m), 6.57 (1H, s), 7.22-7.25 (1H, m), 7.48-7.60 (5H, m), 7.73-7.78 (1H, m), 8.54-8.56 (1H, m). MSp m/z for MH+=368.

### Example 3

### 4-Fluoro-4(pyridine-2-yl)N-[4-trifluoromethylbenzyl]piperidine-1-carboxamide

Using Description Example 4, (4-trifluoromethylbenzyl)isocyanate and the procedure shown in Example 1, the title compound was obtained.

1H NMR δ (ppm) 400 MHz (DMSO): 1.81-1.89 (2H, m), 2.05-2.23 (2H, m), 3.06-3.14 (2H, m), 4.00-4.06 (2H, m), 4.35 (2H, d, J=5.9 Hz), 7.31-7.38 (2H, m), 7.49 (2H, d, J=7.8 Hz), 7.59 (1H, dd, J=1.2, 7.8 Hz), 7.68 (2H, d, J=7.8 Hz), 7.86-7.90 (1H, m), 8.56-8.58 (1H, m). MSp m/z for MH+=382.

### Example 4

### 2-{4-Fluoro-1-[4-trifluoromethylbenzol]piperidin-4-yl}pyridine

4-trifluoromethylbenzoic acid (38 mg, 0.2 mmol) in THF (1 ml) was treated with 1,1′-carbonyldiimidazole (32 mg, 0.2 mmol) and heated at 70° C. for 3 hours. Description Example 4 in THF (1 ml) was added and the reaction mixture was heated at 70° C. overnight. Following evaporation, the compound was purified by column chromatography on silica using 33% ethyl acetate in isohexane as the eluant gave the desired product (25 mg).

1H NMR δ (ppm) 400 MHz (CDCl3) 1.8-2.1 (2H, m), 2.25-2.51 (2H, m), 3.20-3.3.35 (1H, m), 3.34-3.59 (1H, br m), 3.61-3.76 (1H, br m), 4.71-4.85 (1H, br m), 7.24-7.27 (1H, m), 7.57-7.61 (3H, m), 7.70 (2H, d, J=7.8 Hz), 7.74-7.79 (1H, m), 8.57-8.59 (1H, m). MSp m/z for MH+=353.

### Example 5

### 2-(4-Fluoro-1-{[4-trifluoromethylphenyl]acetyl}piperidin-4-yl)pyridine

Using Description Example 4, 4-trifluoromethylphenyl acetic acid and the procedure shown in Example 4, the title compound was obtained.

1H NMR δ (ppm) 500 MHz (CDCl3) 1.86-2.0 (2H, m), 2.13-2.33 (2H, m), 3.03-3.09 (1H, m), 3.46-3.52 (1H, m), 3.83 (2H, d, J 4.2 Hz), 3.85-3.89 (1H, m), 4.65 -4.72 (1H, m), 7.21-7.23 (1H, m), 7.40 (2H, d, J=8.1 Hz), 7.55 (1H, d, J=7.8 Hz), 7.60 (2H, d, J=8.1 Hz), 7.71-7.77 (1H, m), 8.53 (1H, d, J=4.9 Hz). MSp m/z for MH+=367.

### Example 6

### 2-(4-Fluoro-1-{3-[4-trifluoromethylphenyl]propanoyl}piperidin-4-yl)pyridine

Using Description Example 4, 4-trifluoromethylhydrocinnamic acid and the procedure shown in Example 4, the title compound was obtained.

1H NMR δ (ppm) 360 MHz (CDCl3): 1.88-1.96 (2H, m), 2.12-2.33 (2H, m), 2.69 (2H, t, J=7.7 Hz), 2.98-3.09 (3H, m), 3.41-3.49 (1H, m), 3.79-3.84 (1H, m), 4.66-4.71 (1H, m), 7.21-7.24 (1H, m), 7.36 (2H, d, J=8.1 Hz), 7.55 (3H, d, J=8.1 Hz), 7.71-7.76 (1H, m), 8.54 (1H, d, J=3.9 Hz). MSp m/z for MH+=381.

### Example 7

### 4-Fluoro-4-(1-methyl-1H-imidazol-2-yl)-N-[4-trifluoromethylphenyl]piperidine-1-carboxamide

The title compound was prepared in analogous fashion to Example 1 from Description Example 5.

1H NMR δ (ppm) 400 MHz (DMSO): 2.10-2.27 (4H, m), 3.29-3.37 (2H, m), 3.77 (3H, d, J=2.0 Hz), 3.94-4.00 (2H, m), 6.83 (1H, s), 7.17 (1H, s), 7.59 (2H, d J=8.6 Hz), 7.69 (1H, d J=8.6 Hz), 9.01 (1H, s). MSp m/z for MH+=371.

### Example 8

### 4-methoxy-4-pyridin-2-yl-N-[4-trifluoromethylphenyl]piperidine-1-carboxamide

Description Example 6 (250 mg, 0.86 mmol) in dichloromethane (4 ml) was treated with trifluoroacetic acid (1 ml) and stirred at room temperature overnight. The reaction mixture was evaporated, and the amine was isolated using a strong cation exchange cartridge as the free base (186 mg). This amine (83 mg) in dichloromethane (1 ml) was treated with 4-trifluoromethylphenylisocyanate (62 μl, 0.43 mmol) and stirred at room temperature for 1 hour. Following evaporation the compound was purified by column chromatography on silica using 50% ethyl acetate in isohexane as the eluant to give the desired compound (144 mg).

1H NMR δ (ppm) 360 MHz (CDCl3): 2.04-2.25 (4H, m), 3.10 (3H, s), 3.38-3.46 (2H, m), 3.93 (2H, m), 6.54 (1H, s), 7.22 (1H, dd, J=5.1, 7.2 Hz), 7.53 (5H, m), 7.71-7.76 (1H, m), 8.58 (1H, dd, J=0.7, 4.2 Hz). MSp m/z for MH+=380.

### Example 9

### 4-Methoxy-4-pridin-2-yl-N-[4-trifluoromethylbenzyl]piperidine-1-carboxamide

The title compound was obtained using the procedure shown in Example 8 and employing Description Example 6 and [4-trifluoromethylbenzyl]isocyanate.

1H NMR δ (ppm) 360 MHz (CDCl3): 2.02-2.18 (4H, m), 3.07 (3H, s), 3.28-3.36 (2H, m), 3.79-3.83 (2H, m), 4.50 (2H, d, J=5.6 Hz), 4.84-4.87 (1H, m), 7.18-7.22 (1H, m), 7.43 (2H, J=8.1 Hz) 7.49 (1H, J=8 Hz), 7.58 (2H, J=8.1 Hz), 7), 7.69-7.74 (1H, m), 8.58 (1H, dd, J=1.1, 4.2 Hz). MSp m/z for MH+=394.

### Example 10

### 4-Fluoro-N-(4-isopropylphenyl)-4-(3-methylpyridin-2-yl)piperidine-1-carboxamide

4-isopropylphenylisocyanate (40.3 mg, 0.25 mmol) in dichloromethane (1 ml) was treated with Description Example 3 (48.5 mg, 0.25 mmol) in dichloromethane (1 ml) and stirred overnight at room temperature. The compound was purified by column chromatography on silica using 20-40% ethyl acetate in isohexane the eluant to give the desired compound (75 mg).

1H NMR δ (ppm) 400 MHz (CDCl3): 1.23 (6H, d, J=6.7 Hz), 2.10-2.17 (2H, m), 2.35-2.52 (5H, m), 2.83-2.90 (1H, m), 3.37-3.44 (2H, m), 4.02-4.06 (2H, m), 6.35 (1H, s), 7.12-7.16 (3H m), 7.26 (2H, d, J=6.8 Hz), 7.28 (1H, s), 7.48 (1H, dd, J=0.8, 7.0 Hz), 8.35 (1H, d, J=4.7 Hz). MSp m/z for MH+=356.

### Example 11

### 4-Fluoro-4-(3-methylpyridin-2-yl)-N-{4-[1,2,2,2-tetrafluoro-1-trifluoromethylethyl]phenyl}piperidine-1-carboxamide

4-(1,2,2,2-tetrafluoro-1-trifluoromethylethyl)phenylamine (131 mg, 0.5 mmol) in dichloromethane (5 ml) was treated with triphosgene (49 mg, 0.167 mmol) followed by triethylamine (50.5 mg, 0.5 mmol). After one hour Description Example 3 (97 mg, 0.5 mmol) in dichloromethane (1 ml) was added and the reaction mixture was stirred at room temperature overnight. Water (2 ml) was added and the organics were separated using a phase separation cartridge. The compound was purified using a strong cation exchange cartridge followed by column chromatography on silica using 20% ethyl acetate in hexane as the eluant to give the desired compound (15 mg).

1H NMR δ (ppm) 400 MHz (CDCl3): 2.12-2.2 (2H, m), 2.37-2.54 (5H, m)3.42 -3.49 (2H, m), 4.03-4.1 (2H, m), 6.57 (1H, s), 7.15 (1H, dd, J=4.5, 7.6 Hz), 7.49 -7.52 (5H, m). 8.35 (1H, d, J=4.7 Hz). MSp m/z for MH+=482.

### Example 12

### N-(4-Tert-butylphenyl)-4-fluoro-4-(3-methylpyridin-2-yl)piperidine-1-carboxamide

The title compound was obtained using the procedure shown in Example 10 using 4-tert-butylphenyl isocyanate and Description Example 3.

1H NMR δ (ppm) 400 MHz (CDCl3): 1.30 (9H, s), 2.10-2.17 (2H, m), 2.35-2.52 (5H, m), 3.37-3.45 (2H, m), 4.02-4.06 (2H, m), 6.34 (1H, s), 7.14 (1H, dd, J=4.7, 7.8 Hz), 7.26-7.33 (4H, m), 7.48 (1H. d, J=7.8 Hz), 8.35 (1H, d, J=4.3 Hz). MSp m/z for MH+=370.

### Example 13

### 4-Fluoro-4-(3-methyylpyridin-2-yl)-N-[4-(pentafluoro-λ⁶-sulfanyl) phenyl]piperidine-1-carboxamide

The title compound was obtained using the procedure shown in example 17 using [4-(pentafluoro-λ⁶-sulfanyl)phenyl]amine and Description Example 3.

H NMR δ (ppm) 400 MHz (DMSO): 2.06-2.12 (2H, m), 2.17-2.34 (2H, m), 2.48 (3H, d, J=5.5 Hz), 3.19-3.27 (2H, m), 4.1-4.18 (2H, m) 7.28 (1H, dd, J=4.5, 7.6 Hz), 7.63-7.70 (3H, m), 7.75-7.78 (2H, m), 8.37 (1H, d, J=4.5 Hz), 9.10 (1H, s). MSP m/z for MH+=440.

### Example 14

### N-(4-Butyphenyl)-4-fluoro-4-(3-methylpyridin-2-yl)piperidine-1-carboxamide

The title compound was obtained using the procedure shown in Example 10 using 4-n-butylphenyl isocyanate and Description Example 3.

1H NMR δ (ppm) 400 MHz (CDCl3): 0.91 (3H, t, J=7.2 Hz), 1.29-1.39 (2H, m), 1.53-1.61 (2H, m), 2.10-2.17 (2H, m), 2.34-2.58 (7H, m), 3.37-3.44 (2 H, m), 4.02-4.06 (2H, m), 6.35 (1H, s), 7.08-7.14(3H, m), 7.24-7.28 (2H, m), 7.48 (1 H, dd, J =7.0, 0.8 Hz), 8.35 (1H, d, J=4.7 Hz). MSp m/z for MH+=370.

### Example 15

### N-(4-Benzylphenyl)-4-fluoro-4-(3-methylpyridin-2-yl)piperidine-1-carboxamide

The title compound was obtained using the procedure shown in Example 10 using 4-benzylphenyl isocyanate and Description Example 3.

1H NMR δ (ppm) 400 MHz (CDCl3): 2.10-2.17 (2H, m), 2.34-2.48 (2H, m), 2.51 3H, d, J=5.5 Hz), 3.37-3.44 (2H, m), 3.94 (2H, s), 4.01-4.06 (2H, m), 6.37 (1H, s), 7.10-7.20 (6H, m), 7.25-7.30 (4H, m), 7.47 (1H, dd, J=7.4, 0.8 Hz), 8.35 (1H, d, J=4.7 Hz). MSP m/z for MH+=404.

### Example 16

### N-biphenyl-4-yl-4-fluoro-4-(3-methylpyridin-2-yl)piperidine-1-carboxamide

The title compound was obtained using the procedure shown in example 16 using 4-biphenyl isocyanate and Description Example 3.

1H NMR δ (ppm) 400 MHz (DMSO): 2.04-2.13(2H, m), 2.18-2.35 (2H, m), 2.48-2.51(3H, m), 3.20-3.27 (2H, m), 4.13-4.16 (2H, m, 7.26-7.32 (2H, m), 7.40-7.45 (2H, m), 7.55-7.65 (7H, m), 8.38 (1H, d, J=4.7 Hz), 8.73 (1H, s) MSp m/z for MH+=390.

### Example 17

### 4-Fluoro-4-(3-methylpyridin-2-yl)-N-[5-trifluoromethylpyridin-2-yl]piperidine-1-carboxamide

5-Trifluoromethylpyridine-2-carboxylic acid (191 mg, 1 mmol), diphenyl phosphoryl azide (275 mg, 1 mmol) and triethylamine (202 mg, 2 mmol) was heated at 90° C. for 3 hours. Description Example 3 (195 mg, 1 mmol) was added to the reaction mixture and stirred at room temperature for 72 hours. The reaction mixture was evaporated, partitioned between water (2 ml) and dichloromethane (5 ml) in a phase separation cartridge. The aqueous phase was washed with more dichloromethane and the combined organics were evaporated. The compound was purified by mass-triggered HPLC to give the desired compound (27 mg).

1H NMR δ (ppm) 040 MHz (CDCl3): 2.12-2.22 (2H, m), 2.36-2.52 (5H, m), 3.42-3.50 (2H, m), 4.07-4.13 (2H, m), 7.14 (1H, dd, J=4.5, 7.6 Hz), 7.43-7.51 (2H, m), 7.87 (1H, dd, J=2.3, 8.6 Hz), 8.19 (1H, d, J=9.0 Hz), 8.35 (1H, d, J=4.7 Hz), 8.46 (1H, s). MSp m/z for MH+=383.

### Example 18

### 4-(3-Chloropyridin-2-yl)-4-fluoro-N-[4-trifluoromethphenyl]piperidine-1-carboxamide

1,4-diazabicyclo[2.2.2]octane (3.08 g, 27.5 mmol) in diethyl ether (110 ml) was treated with n-butyl lithium in hexanes (17.2 ml, 1.6 M, 25 mmol) at −40° C. keeping the temperature in the range −40° C. to −30° C., and stirred in this temperature range for 1 hour. The reaction was cooled to −65° C. and 3-chloropyridine (2.84 g, 25 mmol) was added and stirred at this temperature for 1 hour. The mixture was cooled to −78° C. and 1-tertbutoxycarbonyl-4-piperidone (4.98 g, 25 mmol) in diethyl ether (50 ml) was added dropwise. After 1 hour at −78° C., the reaction was allowed to warm to −50° C. over 2 hours. Saturated aqueous ammonium chloride (50 ml) was added and the aqueous was extracted using ethyl acetate (4×50 ml). The combined organics were dried over sodium sulphate and evaporated. Column chromatography on silica using 5-20% ethyl acetate in isohexane as eluant gave tert-butyl 4-hydroxy-4-(3-chloropyridin-2-yl)piperidine-1-carboxylate contaminated with 1-tertbutoxycarbonyl-4-piperidone (3.31 g). This mixture (1.9 g) was converted to the desired compound (510 mg) using the chemistry described in Description Example 2 and Example 1.

1H NMR δ (ppm) 400 MHz (CDCl3) 2.32-2.50 (4H, m), 3.45-3.52 (2H, m), 4.02-4.09 (2H, m), 6.57 (1H, s), 7.22-7.26 (1H, m), 7.48-7.56 (4H, m), 7.76 (1H, dd, J=1.4, 8.0 Hz), 8.44-8.46 (1H, m). MSp m/z for MH+=402.

### Example 19

### 4-Fluoro-4-(3-fluoropyridin-2-yl)-N-[4-trifluoromethylphenyl]piperidine-1-carboxamide

The title compound was obtained using the chemistry described in Example 18, using 3-fluoropyridine as the starting material.

H NMR δ (ppm) 400 MHz (CDCl3): 2.27-2.45 (4H, m), 3.46-3.53 (2H, m), 4.02-4.07 (2H, m), 6.57 (1H, s), 7.31-7.38 (1H, m), 7.44-7.56 (5H, m), 8.39 (1H, d, J=4.7 Hz). MSP m/z for MH+=386.

### Example 20

### 4-Fluoro-4-(3-methoxypyridin-2-yl)-N-[4-trifluoromethylphenyl]piperidine-1-carboxamide

The title compound was prepared from 2-bromo-3-methoxypyridine using the chemistry described for Description Example 1 and Example 1.

1H NMR δ (ppm) 400 MHz (CDCl3): 2.35-2.48 (4H, m), 3.46-3.53 (2H, m), 3.89 (3H, s) 3.98-4.03 (2H, m), 6.57 (1H, s), 7.27 (2H, d, J=2.7 Hz), 7.49-7.54 (4H, m), 8.17-8.2 (1H, m). MSp m/z for MH+=398.

### Example 21

### 4-Fluoro-4-(3-methylpyridin-2-yl)-N-[4-trifluoromethylphenyl]piperidine-1-carbothioamide

The title compound was prepared using the procedure described in Example 10 with Description Example 3) and 4-trifluoromethylphenyl isothiocyanate.

1H NMR δ (ppm) 400 MHz (CDCl3): 2.14-2.21 (2H, m), 2.47-2.65 (5H, m), 3.60-3.67 (2H, m), 4.52-4.58 (2H, m), 7.15 (1H, dd, J=4.7, 7.4 Hz), 7.23 (1H, s), 7.27 (2H, d, J=8.2 Hz), 7.49 (1H, dd, J=7.0, 0.8 Hz), 7.60 (2H, d, J=8.2 Hz), 8.35 (1H, d, J=4.7 Hz). MSp m/z for MH+=398.

### Example 22

### N-cyano-4-fluoro-4-(3-methylpyridin-2-yl)-N-[4-trifluoromethyl phenyl]piperidine-1-carboximidamide

Example 21 (39.7 mg 0.1 mmol), cyanamide (310 mg, 7.4 mmol) and silver acetate (17 mg, 0.1 mmol) in acetonitrile (3 ml) were heated in a pressurized microwave reactor at 140° C. for 10 minutes. The reaction was repeated on twice this scale and the reactions combined. The reactions were combined, evaporated and partitioned between dichloromethane (50 ml) and water (50 ml). The organics were washed with water (50 ml), dried (Na₂SO₄) and evaporated. The desired compound (20 mg) was obtained by column chromatography purification on silica using 25%-100% ethyl acetate in isohexane as the eluant.

1H NMR δ (ppm) 400 MHz (CDCl3): 2.04-2.17 (2H, m), 2.34-2.54 (5H, m), 3.35-3.43 (2H, m), 3.90-3.95 (2H, m), 7.10-7.16 (3H, m), 7.18(1H s) 7.47 (1H d J=7 Hz) 7.62 (2H, d, J=8.2 Hz). 8.34 (1H, d, J=4.7 Hz) MSp m/z for MH+=406.

### Example 23

### 4-Fluoro-4-(3-methylpyridin-2-yl)-N-(1-phenylpiperidin-4-yl)-N-[4-trifluoromethylphenyl]piperidine-1-carboximidamide

Example 21 (397 mg, 1 mmol), 1-phenylpiperidin-4-ylamine (176 mg, 1 mmol) and silver acetate (167 mg, 1 mmol) in acetonitrile (20 ml) were heated at reflux overnight. The reaction mixture was evaporated, purified by column chromatography on silica using 2-10% methanol in dichloromethane as the eluant, followed by mass triggered HPLC purification using an acid based eluant. The free base was liberated using a strong cation exchange cartridge to give the desired compound (40 mg).

1H NMR δ (ppm) 400 MHz (CDCl3), 1.45-1.52 (2H, m), 2.00-2.13 (4H, m), 2.32-2.52 (5H, m), 2.74-2.80 (2H, m), 3.25-3.31 (2H, m), 3.37-3.46 (1H, m), 3.55-3.76 (4H, m), 6.82-6.93 (5H, m), 7.14 (1H, dd, J=4.7, 7.4 Hz), 7.23-7.27 (2H, m), 7.48-7.52 (3H, m), 8.37 (1H, d, J=4.3 Hz). MSp m/z for MH+=540.

### Example 24

### 4-Fluoro-4-phenyl-N-[4-trifluoromethylphenyl]piperidine-1-carboxamide

Using 4-fluoro-4-phenylpiperidine, 4-trifluoromethylphenylisocyanate and the procedure in Example 10 the title compound was obtained.

1H NMR δ (ppm) 400 MHz (MeOD): 1.98-2.24 (4H, m), 3.34-3.38 (2H, m), 4.19-4.24 (2H, m), 7.28-7.32 (1H, m), 7.38 (2H, m), 7.43 (2H, m), 7.54 (2H, d, J=8.8 Hz), 7.59 (2H, d, J=8.7 Hz). MSp m/z for MH+=367.

### Example 25

### (+/−)-(syn)-4-fluoro-2-methyl-4-(3-methylpyridin-2-yl)-N-[4-trifluoromethylphenyl]piperidine-1-carboxamide

Step 1: Zinc powder (48 g, 734 mmoles) was added in a single portion to a stirred solution of 2-methyl-4-oxo-3,4-dihydro-2H-pyridine-1-carboxylic acid methyl ester (11.38 g, 67.3 mmoles) (Curmins, D. L. and Al-awar, R. S., Journal of Organic Chemistry, 1995, 60, 711-716 using methylmagnesium bromide as the Grignard reagent) in acetic acid (70 ml) at room temperature. The reaction was then heated at reflux for 18 hours, cooled, the zinc filtered and washed with acetic acid. The resulting filtrate was evaporated in vacuo to give an orange oil. This was absorbed onto silica and purified by column chromatography on silica using 25% ethyl acetate in isohexane to give 2-methyl-4-oxopiperidine-1-carboxylic acid methyl ester as a clear oil (5.3 g, 46% yield).

1H NMR δ (ppm) 400 MHz (DMSO): 1.10 (3H, d, J=7.0 Hz), 2.15-2.28 (2H, m), 2.43-2.48 (1H, m), 2.74 (1H, dd, J=6.7, 14.9 Hz), 3.34-3.38 (1H, m), 3.64 (3H, s), 4.02-4.05 (1H, m), 4.50-4.54 (1H, m). MSp m/z for MH+=172.

Step 2: Methyl 4-hydroxy-2-methyl-4-(3-methylpyridin-2-yl)piperidine-1-carboxylate was synthesized in the same manner as Description Example 1 using the product of Step 1 and 2-bromo-3-methylpyridine.

1H NMR δ (ppm) 400 MHz (DMSO): 1.35 (3H, d, J=7.0 Hz), 1.76-1.83 (2H, m), 1.98-2.14 (2H, m), 2.56 (3H, s), 3.32-3.35 (1H, m), 3.60 (3H, s), 3.85-3.91 (1H, m), 4.28-4.35 (1H, m), 5.28 (1H, s), 7.15-7.18 (1H, m), 7.52-7.54 (1H, m), 8.29 (1H, dd, J=6, 1.3 Hz). MSp m/z for MH+=265.

Step 3: 47% hydrogen bromide in acetic acid (3 ml, excess) was added dropwise to a stirred solution of the product of Step 2 (361 mg, 1.4 mmoles) in acetic acid (1 ml) and stirred at room temperature for 48 hours. The reaction was concentrated in vacuo to give a pale brown solid. This was then reacted with 4-trifluoromethylphenylisocyanate using the same procedure as for Example 1 to give crude material which was purified by mass-triggered HPLC using an acid based eluant, and strong cation exchange cartridge to give (+/−)-(syn)-4-hydroxy-2-methyl-4-(3-methylpyridin-2-yl)-N-[4-trifluoromethylphenyl]piperidine-1-carboxamide free base as a white solid (256 mg, 46%).

1H NMR δ (ppm) 400 MHz (DMSO): 1.42 (3H, d, J=6.7 Hz), 1.84-1.87 (2H, m), 2.09-2.16 (1H, m), 2.20-2.34 (1H, m), 2.58 (3H, s), 3.38-3.46 (1H, m), 3.96-4.00 (1H, m), 4.50-4.53 (1H, m), 5.31 (1H, s), 7.16-7.19 (1H, m), 7.53-7.57 (3H, m), 7.69 (2H, d, J=9.6 Hz), 8.31 (1H, dd, J=3.9, 0.8 Hz), 8.81 (1H, m). MSp m/z for MH+=394.

Step 4: To a suspension of the product of Step 3 (221 mg, 0.56 mmol) in dichloroethane (10 ml) at −78° C. diethylaminosulphur trifluoride (144 μl, 1.18 mmol) was added dropwise. Workup was analogous to Description Example 2, to give crude product which was purified via column chromatography on silica using 10-20% ethyl acetate in isohexane to give the desired product (8 mg, 4%).

1H NMR δ (ppm) 500 MHz MeOD): 1.15 (3H, d, J=6.6 Hz), 2.21-2.49 (3H, m), 2.50 (2H, s), 2.51 (3H, d, J=4.8 Hz), 2.58-2.67 (1H, m), 3.53-3.59 (1H, m), 4.06-4.11 (1H, m), 4.38-4.45 (1H, m), 7.24 (1H, dd, J=4.7, 7.6 Hz), 7.54 (2H, d, J=8.7 Hz), 7.60 (2H, d, J=8.8 Hz), 8.36 (1H, d, J=4.6 Hz). MSp m/z for MH+=396.

### Example 26

### 4-(Fluoromethyl)-4-pyridin-2-yl-N-[4-trifluoromethylphenyl]piperidine-1-carboxamide

Step 1: Sodium hydride (60% in mineral oil, 2.04 g, 51 mmol) was added portion wise over 8 minutes to a stirring solution of 2-pyridineacetonitrile (1.8 ml, 17 mmol) and N-(tert-butyloxycarbonyl)bis(2-chloroethyl)amine in anhydrous DMF (50 ml) at 0° C. The reaction was then heated at 60° C. for 5½ hours. The reaction was cooled and extracted into ethyl acetate (4×150 ml), and washed with water (3×200 ml). The organic layer was then dried over anhydrous MgSO₄, filtered and evaporated in vacuo to give a red/black oil. This was absorbed onto silica and purified by column chromatography using 20% ethyl acetate in isohexane to give tertbutyl 4-cyano-4-pyridin-2-ylpiperidine-1-carboxylate as an orange solid (2.13 g, 44%).

1H NMR δ (ppm) 360 MHz (CDCl3): 1.48 (9H, s), 2.04-2.08 (2H, m), 2.17-2.25 (2H, m), 3.15-3.28 (2H, m), 4.20-4.35 (2H, m), 7.25-7.29 (1H, m), 7.61 (1H, d, J=8 Hz), 7.73-7.78 (1H, m), 8.61 (1H, dd, J=0.7, 3.9 Hz). MSp m/z for MH+=287 (−56).

Step 2: HCl gas was bubbled through a solution of the product of Step 1 (1 g, 3.5 mmol) in anhydrous methanol (20 ml) for 10 minutes at 0° C. This was warmed to room temperature and stirred for 48 hours, then evaporated in vacuo. The resulting solid (1.03 g, 3.5 mmol) was suspended in anhydrous dichloroethane (25 ml). To the stirring suspension at 0° C. triethylamine (1.1 ml, 7.7 mmol), benzaldehyde (400 μl, 3.85 mmol) and 3 Å molecular sieves (5 g) were added and allowed to stirred at room temperature for 15 minutes before sodium cyanoborohydride (242 mg, 3.85 mmol) was added in a single portion and the solution stirred at room temperature for 18 hours. The solvent was then evaporated in vacuo, to the residue water (50 ml) was added and extracted into ethyl acetate (2×50 ml). The organic layer was dried over anhydrous MgSO₄, filtered and evaporated in vacuo to give an orange oil. This was purified by column chromatography on silica using 0-5% methanol in dichloromethane as eluant. This gave methyl 1-benzyl-4-pyridin-2-ylpiperidine-4-carboxylate as an orange oil (820 mg, 76%).

1H NMR δ (ppm) 360 MHz (CDCl3): 2.10-2.1 (2H, m), 2.23-2.29 (2H, m), 2.49 -2.54 (2H, m), 2.74-2.77 (2H, m), 3.47 (2H, s), 3.69 (3H, s), 7.16 (1H, dd, J=5.1, 7.2 Hz), 7.28-7.34 (6H, m), 7.64 (1H, td, J=7.7, 1.7 Hz), 8.56 (1H, dd, J=0.7, 3.9 Hz) MSp m/z for MH+=311.

Step 3: Lithium aluminium hydride (1 M in tetrahydrofuran, 1 ml, 1 mmol) was added to a stirred solution of the product of Step 2 (320 mg, 1 mmol) in anhydrous THF (3 ml) at −30° C. and stirred at this temperature for 1 hour. Water (2 ml) was added then extracted into ethyl acetate (2×5 ml) organic layers combined, dried over anhydrous MgSO₄, filtered, filtrate evaporated in vacuo to give (1-benzyl-4-pyridin-2-ylpiperidin-4-yl)methanol as an orange oil which solidified on standing (263 mg, 93%).

1H NMR δ (ppm) (CDCl3): 1.89-1.94 (2H, m), 2.14-2.21 (2H, m), 2.43-2.56 (4H, m), 3.51 (2H, s), 3.80 (2H, s), 7.14-7.17 (1H, m), 7.19-7.34 (5H, m), 7.37 (1H, d, J=8.1 Hz), 7.66-7.71 (1H, m), 8.52 (1H, dd, J=0.7, 3.9 Hz). MSp m/z for MH+=283.

Step 4: A slurry of 10% palladium on carbon (84 mg) was added to a solution of the product of Step 3 (124 mg, 0.44 mmol) in ethanol (10 ml). This was hydrogenated at room temperature under atmospheric pressure for 72 hours. The catalyst was filtered, washed with ethanol, and the filtrate concentrated in vacuo. The resulting amine (85 mg, 0.44 mmol) was dissolved in dichloromethane (10 ml), to the stirring solution di-tert-butyldicarbonate (95 mg, 0.44 mmol) added 5 and stirred at room temperature for 2 hours. The reaction was evaporated in vacuo, extracted into ethyl acetate and washed with water, then the organic layer dried over MgSO₄, filtered and evaporated to give tert-butyl 4-hydroxymethyl4-pyridin-2-ylpiperidine-1-carboxylate as an off-white solid (120 mg, 93%).

1H NMR δ (ppm) 400 MHz (CDCl3): 1.46 (9H, s), 1.80-1.86 (2H, m), 2.07-2.14 (2H, m), 3.43-3.49 (4H, m), 3.80 (2H, bs), 3.94 (1H, bs), 7.16-7.21 (1H, m), 7.33 (1H, d, J=8.2 Hz), 7.68-7.73 (1H, m), 8.53-8.57 (1H, m). MSp m/z for MH+=237 (−56).

Step 5: Diethylaminosulphur trifluoride (76 μl, 0.62 mmol) was added to a stirring solution of the product of Step 4 (120 mg, 0.41 mmol) in anhydrous ethyl acetate (3 ml) at −78° C. The reaction was allowed to warm to RT overnight. The solution was evaporated and to the resulting oil tetrabutylammonium fluoride (1 M solution in tetrahydrofuran, 820 μl 0.82 mmol) added and refluxed for 2 hours. The reaction was cooled to RT water (3 ml) added and the aqueous layer was 20 extracted with ethyl acetate (3×5 ml). The combined organics were dried over MgSO₄, filtered and the filtrate evaporated in vacuo. The resulting oil was stirred in trifluoroacetic acid (1 ml) for 1 hour, the solvent then evaporated and the residue dissolved in dichloromethane. To this stirring solution triethylamine (123 μl, 0.90 mmol), and 4-trifluoromethylphenylisocyanate (59 μl, 0.41 mmol) were added and the solution stirred at room temperature for 18 hours. The reaction was evaporated, water (5 ml) added and extracted with ethyl acetate (3×5 ml). The combined organics were evaporated to give an oily orange compound. This was washed with dichloromethane and purified on a silica prep plate using 5% methanol in dichloromethane with 0.1% ammonia as the eluant. This gave the desired compound as a white solid (1.8 mg, 1%).

1H NMR δ (ppm) 500 MHz (MeOD): 1.89-1.94 (2H, m), 2.48-2.51 (2H, m), 3.08-3.15 (2H, m), 3.92-3.97 (2H, m), 4.45 (2H, d, J=48 Hz), 7.28-7.30 (1H, m), 7.51-7.67 (5H, m), 7.80-7.84 (1H, m), 8.60-8.61 (1H, m). MSp m/z for MH+=382.

### Examples 27 and 28

### Syn- and Anti-3-fluoro-3-pyridin-2-yl-N-[4-trifluoromethyl phenyl]-8-azabicyclo[3.2.1]octane-8-carboxamide & 3-fluoro -3-pyridin-2-yl-N-[4-trifluoromethylphenyl]-8-azabicyclo [3.2.1]octane-8-carboxamide

Step 1: 2-Lithiopyridine was added to tert-butoxycarbonyltropinone according to the procedure in Description Example 1 to give 3-hydroxy-3-pyridin-2-yl-8-azabicyclo[3.2.1]octane-8-carboxylic acid tert-butyl ester. Without complete purification, the product was treated according to the procedure in Description Example 2 to give 3-fluoro-3-pyridin-2-yl-8-azabicyclo[3.2.1]octane-8-carboxylic acid tert-butyl ester as a 1.2:1 mixture of syn: anti isomers. Syn: 1H NMR δ (ppm) 500 MHz (CDCl₃): 1.50 (9H, s), 1.88-2.19 (4H, m), 2.33 (2H, m), 2.68 (2H, m), 4.47 (2H, m), 7.15 (1H, m), 7.49 (1H, m), 7.67 (1H, m), 8.52 (1H, m). MSp m/z for MH+=307. Anti. 1H NMR δ (ppm) 500 MHz (CDCl₃): 1.51 (9H, s), 1.93 (2H, m), 2.33 (2H, m), 2.59 (4H, m), 4.47 (2H, m), 7.22 (1H, m), 7.62 (1H, m), 7.71 (1H, m), 8.56 (1H, m). MSp m/z for MH+=307.

Step 2a: The major isomer of the product of Step 1 was treated according to the procedure in Example 1 to give the title compound (Example 27). 1H NMR δ (ppm) 700 MHz (CDCl₃): 2.07 (2H, d, J 16.8 Hz), 2.14 (2H, m), 2.33 (2H, dd, J 14.0, 6.3 Hz), 2.75 (2H, m), 4.51 (2H, brs), 6.58 (1H, s), 7.21 (1H, m), 7.50 (1H, m), 7.56 (4H, m), 7.73 (1H, m), 8.53 (1H, m). MSp m/z for MH+=394.

Step 2b: The minor isomer of the product of Step 1 was treated according to the procedure in Example 1 to give the title compound (Example 28). 1H NMR δ (ppm) 700 MHz (CDCl₃): 1.80 (2H, dd, J 14.0, 6.3 Hz), 2.07 (2H, dd, J 7.0, 3.5 Hz), 2.60 (4H, m), 4.48 (2H, brs), 6.56 (1H, s), 7.25 (1H, m), 7.55 (2H, d, J 9.1 Hz), 7.57 (2H, d, J 9.1 Hz), 7.61 (1H, m), 7.74 (1H, m), 8.58 (1H, m). MSp m/z for MH+=394.

### Example 29

### 4-Fluoro-4-pyrimidin-2-yl-N-[4-trifluoromethylphenyl]piperidine-1-carboxamide

Step 1: 4-Hydroxy-4-pyrimidin-2-ylpiperidine-1-carboxylic acid tert-butyl ester was prepared according to WO-A-9903847 and then treated according to the procedure in Description Example 2 to give 4-fluoro-4-pyrimidin-2-ylpiperidine-1-carboxylic acid tertbutyl ester.

1H NMR δ (ppm) 400 MHz (CDCl₃): 1.48 (9H, s), 2.08-2.34 (4H, m), 3.27 (2H, m), 4.08 (2H, m), 7.27 (1H, t, J 4.7 Hz), 8.79 (2H, d, J 4.7 Hz).

Step 2: The product of Step 1 was treated according to the procedure in Example 1 to give the title compound.

1H NMR δ (ppm) 500 MHz (CDCl_{3l):} 2.25-2.44 (4H, m), 3.49 (2H, m), 4.07 (2H, m), 6.56 (1H, s), 7.29 (1H, t, J 4.8 Hz), 7.49 (2H, d, J 8.7), 7.55 (2H, d, J 8.7), 8.81 (2H, d, J 4.8 Hz). MSp m/z for MH+=369.

### Example 30

### 4-Fluoro-4-(3-phenylpropyl)-N-[4-trifluoromethylphenyl]piperidine-1-carboxamide

The title compound was prepared using a procedure analogous to that in Example 7 using 4-fluoro-4-(3-phenylpropyl)piperidine hydrochloride as the amine.

1H NMR δ (ppm) 400 MHz (DMSO): 1.43-1.82 (8H, m), 2.53-2.63 (2H, m), 3.03-3.10 (2H, m), 3.92-3.98 (2H, mz), 7.14-7.23 (3H, m), 7.26-7.30 (2H, m), 7.57 (2H, d J=8.6 Hz), 7.68 (2H, d J=8.6 Hz), 8.91 (1H, s). MSp m/z for MH+=409.

### Example 31

### 2-[4-Fluoro-4-(3-methylpyridin-2-yl)piperidin-1-yl]-6-trifluoromethyl-1H-benzimidazole

Description Example 3 (87.2 mg, 0.46 mmol) was treated with ethanol (1 ml) and 2-chloro-5-(trifluoromethyl)-1H-benzimidazole (100 mg, 0.46 mmol) (WO-A-021471) and heated in a pressurized microwave vessel at 160° C. for 10 minutes. After evaporation, the compound was purified by column chromatography using 2-5% methanol in dichloromethane as the eluant to give 2-[4-hydroxy-4-(3-methylpyridin-2-yl)piperidin-1-yl]-6-trifluoromethyl-1H-benzimidazole (18 mg) MSp m/z for MH+=377). This was converted to the title compound using the procedure outlined in Description Example 2.

1H NMR δ (ppm) 400 MHz (DMSO): 1.9-2.05 (2H, m), 2.508-2.27(2H, m), 2.32-2.34(3H, m), 3.20-3.32 (2H, m), 3.96-4.03 (2H, m), 7.04-7.21 (3H, m), 7.23-7.35 (1H, m), 7.48 (1H, d J=6.7 Hz), 8.17 (1H, d J=4.3 Hz), 11.67-11.72 (1H, m). MSp m/z for MH+=379.

### Example 32

### 2-(4-Fluoro-4-pyridin-2-ylpiperidin-1-yl)-6-(trifluoromethyl)-1H-benzimidazole

The title compound was prepared using a procedure analogous to that in Example 31 from Description Example 4.

1H NMR δ (ppm) (CDCl3):, 2.02-2.07 (2H, m), 2.41-2.59 (2H, m), 3.56-3.61 (2H, m), 4.10-4.13 (2H, m), 7.22-7.59 (6H, m), (7.73-7.79 (1H, m), 8.52-8.53 (1H, m). MSp m/z for MH+=365.

### Example 33

### 4-Fluoro-N-[4-trifluoromethylphenyl]-4-[3-trifluoromethylpyridin-2-yl]piperidine-1-carboxamide

2-bromo-3-trifluoromethylpyridine (1.13 g, 5 mmol) and 1-butoxycarbonyl-4-piperidone (1 g, 5 mmol) in THF(20 ml) was cooled to −78° C. and tert-butyffithium (4.4 ml, 1.7 M in pentane, 7.5 mmol) was added dropwise to this solution. After stirring at this temperature for 30 minutes, the reaction mixture was allowed to warm to room temperature over two hours. Water (50 ml) was added and the aqueous was extracted using ethyl acetate (3×50 ml). The combined organics were dried over sodium sulphate and evaporated. Column chromatography on silica using 10-20% ethyl acetate in isohexane as eluant gave tert-butyl 4-hydroxy-4-(3-trifluoromethylpyridin-2-yl)piperidine-1-carboxylate contaminated with 1-butoxycarbonyl-4-piperidone (650 mg). This mixture was converted to the desired compound using the chemistry described in Description Example 2 and Example 1.

1H NMR δ (ppm) 400 MHz (CDCl3) 2.13-2.05 (2H, m), 2.60-2.42 (2H, m), 3.46-3.40 (2H, m), 4.11 (2H, dd, J=4.8, 13.4 Hz), 6.61 (1H, s), 7.42-7.36 (1H, m), 7.48-7.57 (4H, m), 8.11 (1H, d, J=8.0 Hz), 8.70 (1H, d, J=4.6 Hz). MSp m/z for MH+=436.

Examples 34 to 39 were made using the procedure shown in Example 10 using Description Example 3 and 4-tolyl isocyanate, 4-ethylphenyl isocyanate, 4-chlorophenyl isocyanate, 4-trifluoromethoxy isocyanate, 4-cyanophenyl isocyanate and 4-dimethylaminophenyl isocyanate respectively.

### Example 34

### 4-Fluoro-N-(4-methylphenyl)-4-(3-methylpyridin-2-yl)piperidine-1-carboxamide

1H NMR δ (ppm) 400 MHz (CDCl3): 2.11-2.17 (2H, m), 2.30 (3H, s), 2.50 (5H, m), 3.38-3.44 (2H, m), 4.03-4.15 (2H, m), 6.34 (1H, s), 7.10-7.14 (3H, m), 7.23-7.26 (2H, m), 7.48 (1H, d, J=8 Hz), 8.35 (1H, d, J=4.6 Hz). MSp m/z for MH+=328.

### Example 35

### N-(4-ethylphenyl)-4-fluoro-4-(3-methylpyridin-2-yl)piperidine-1-carboxamide

1H NMR δ (ppm) 360 MHz (CDCl3): 1.21 (3H, t, J=7.6 Hz), 2.11-2.17 (2H, m), 2.34-2.52 (5H, m),2.6 (2H, q, J=7.6 Hz) 3.37-3.45 (2H, m), 4.02-4.08 (2H, m), 6.36 (1H, s), 7.12-7.14 (3H, m),7.25-7.28 (2H, m) 7.48 (1H, d, J=7.0 Hz), 8.36 (1H, d, J=4.6 Hz). MSp m/z for MH+=342.

### Example 36

### N-(4-chlorophenyl)-4-fluoro-4-(3-methylpyridin-2-yl)piperidine-1-carboxamide

1H NMR δ (ppm) 360 MHz (CDCl3): 2.12-2.18 (2H, m), 2.34-2.52 (5H, m), 3.38-3.46 (2H, m), 4.04 (2H, d, J=13.4 Hz), 6.42 (1H, s), 7.14 (1H, dd, J=4.7, 7.7 Hz), 7.24-7.28 (2H, m), 7.30-7.34 (2H, m) 7.49 (1H, d, J=7.7 Hz), 8.35 (1H, d, J=4.7 Hz).). MSp m/z for MH+=348.

### Example 37

### 4-Fluoro-4-(3-methylpyridin-2-yl)-N-[4-trifluoromethoxyphenyl]piperidine-1-carboxamide

1H NMR δ (ppm) 360 MHz (CDCl3): 2.12-2.20 (2H, m), 2.34-2.53 (5H, m), 3.39-3.47 (2H, m), 4.01-4.09 (2H, m), 6.46 (1H, s), 7.12-7.18 (3H, m), 7.37-7.41 (2H, m), 7.49 (1H, d, J=7.7 Hz), 8.36 (1H, d, J=4.8 Hz). MSp m/z for MH+=398.

### Example 38

### N-(4-cyanophenyl)-4-fluoro-4-(3-methylpyridin-2-yl)piperidine-1-carboxamide

1H NMR δ (ppm) 360 MHz (CDCl3): 2.11-2.22 (2H, m), 2.35-2.53 (5H, m), 3.41-3.49 (2H, m), 4.02-4.11 (2H, in), 6.68 (1H, s), 7.15 (1H, dd, J=4.7, 7.7 Hz), 7.49-7.59 (5H, m), 8.35 (1H, d, J=4.6 Hz). MSp m/z for MH+=339.

### Example 39

### N-[4-dimethylaminophenyl]-4-fluoro-4-(3-methylpyridin-2-yl) piperidine-1-carboxamide

1H NMR δ (ppm) 400 MHz (DMSO): 2.01-2.08 (2H, t, J=12.1 Hz), 2.16-2.33 (2H, m), 2.49 (3H, d J=5.4 Hz), 2.87 (6H, s), 3.12-3.25 (2H, m), 4.06-4.16 (2H, m), 6.74 (2H, s), 7.27-7.31 (5H, m), 7.66 (1H, d, J=6.9 Hz), 8.33-8.41 (2H, m). MSp m/z for MH+=357.

1-9. (canceled)

10. A compound of formula (I): 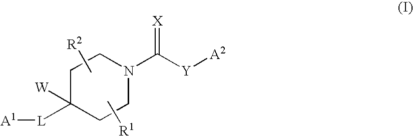
wherein:
A¹ is phenyl, a six-membered aromatic heterocycle containing one, two or three nitrogen atoms, or a five-membered aromatic heterocycle containing up to four heteroatoms chosen from O, N and S, at most one heteroatom being O or S;
A¹ is unsubstituted or substituted by one, two or three substituents independently chosen from halogen, C₁₋₆alkyl, C₂₋₆alkenyl, C₂₋₆alkynyl, haloC₁₋₆alkyl, C₁₋₆alkoxy, haloC₁₋₆alkoxy, hydroxy, cyano, nitro and amino;
A² is phenyl, a six-membered aromatic heterocycle containing one, two or three nitrogen atoms, or a five-membered aromatic heterocycle containing up to four heteroatoms chosen from O, N and S, at most one heteroatom being O or S;
A² is unsubstituted or substituted by one, two or three groups independently chosen from halogen, cyano, nitro, amino, C₁₋₆alkylamino, di(C₁₋₆alkyl)amino, C₁₋₆alkyl C₂₋₆alkenyl, C₂₋₆alkynyl, haloC₁₋₆alkyl, hydroxy, C₁₋₆alkoxy, haloC₁₋₆alkyl, thiol, SF₅, phenylC₁₋₆alkyl and phenyl;
L is a bond or C₁₋₆alkylene;
R¹ and R² independently chosen from hydrogen and C₁₋₆alkyl;
or R¹ and R² may, together, form a methylene or ethylene bridge;
W is halogen, C₁₋₆alkyl, haloC₁₋₆alkyl, C₁₋₆alkoxy or haloC₁₋₆alkoxy;
X is O, S or NR³ where R³ is hydrogen, hydroxy, C₁₋₆alkoxy, C₁₋₆alkyl, cyano, C₃₋₆cycloalkyl, a six-membered saturated heterocycle containing one or two heteroatoms independently chosen from O, N and S, and R³ is, if possible, optionally substituted by C₁₋₆alkyl, C₁₋₆alkoxy, haloC₁₋₆alkyl, haloC₁₋₆alkoxy, halogen, amino, nitro, hydroxy, phenyl, a six-membered aromatic heterocycle containing up to three nitrogen atoms or a five-membered aromatic heterocycle containing up to four heteroatoms chosen from O, N and S, at most one heteroatom being O or S;
or X, together with the atom to which it is attached, and Y, form an unsaturated five-membered ring together with A²;
Y is a bond, C₁₋₄alkylene, NH or NH(CH₂)₁₋₃;
or a pharmaceutically acceptable salt thereof.

11. A compound selected from:
4-fluoro-4-(3-methylpyridin-2-yl)-N-[4-trifluoromethylphenyl]piperidine-1-carboxamide;
4-fluoro-4(pyridin-2-yl)N-[4-trifluoromethylphenyl]piperidine-1-carboxamide;
4-fluoro-4(pyridine-2-yl)N-[4-trifluoromethylbenzyl]piperidine-1-carboxamide;
2-{4-fluoro-1-[4-trifluoromethylbenzoyl]piperidin-4-yl}pyridine;
2-(4-fluoro-1-{[4-trifluoromethylphenyl]acetyl}piperidin-4-yl)pyridine;
2-(4-fluoro-1-{3-[4-trifluoromethylphenyl]propanoyl}piperidin-4-yl)pyridine 4-fluoro-4-(1-methyl-1H-imidazol-2-yl)-N-[4-trifluoromethylphenyl]piperidine-1-carboxamide;
4-methoxy-4-pyridin-2-yl-N-[4-trifluoromethylphenyl]piperidine-1-carboxamide;
4-methoxy-4-pyridin-2-yl-N-[4-trifluoromethylbenzyl]piperidine-1-carboxamide;
4-fluoro-N-(4-isopropylphenyl)-4-(3-methylpyridin-2-yl)piperidine-1-carboxamide;
4-fluoro-4-(3-methylpyridin-2-yl)-N-{4-[1,2,2,2-tetrafluoro-1-trifluoromethylethyl]phenyl}piperidine-1-carboxamide;
N-(4-Tert-butylphenyl)-4-fluoro-4-(3-methylpyridin-2-yl)piperidine-1-carboxamide;
4-fluoro-4-(3-methylpyridin-2-yl)-N-[4-(pentafluoro-λ⁶-sulfanyl)phenyl]piperidine-1-carboxamide;
N-(4-Butylphenyl)-4-fluoro-4-(3-methylpyridin-2-yl)piperidine-1-carboxamide;
N-(4-Benzylphenyl)-4-fluoro-4-(3-methylpyridin-2-yl)piperidine-1-carboxamide;
N-biphenyl-4-yl-4-fluoro-4-(3-methylpyridin-2-yl)piperidine-1-carboxamide;
4-fluoro-4-(3-methylpyridin-2-yl)-N-[5-trifluoromethylpyridin-2-yl]piperidine-1-carboxamide;
4-(3-chloropyridin-2-yl)-4-fluoro-N-[4-trifluoromethylphenyl]piperidine-1-carboxamide 4-fluoro-4-(3-fluoropyridin-2-yl)-N-[4-trifluoromethylphenyl]piperidine-1-carboxamide;
4-fluoro-4-(3-methoxypyridin-2-yl)-N-[4-trifluoromethylphenyl]piperidine-1-carboxamide;
4-fluoro-4-(3-methylpyridin-2-yl)-N-[4-trifluoromethylphenyl]piperidine-1-carbothioamide;
N′-cyano-4-fluoro-4-(3-methylpyridin-2-yl)-N-[4-trifluoromethylphenyl]piperidine-1-carboximidamide;
4-fluoro-4-(3-methylpyridin-2-yl)-N′-(1-phenylpiperidin-4-yl)-N-[4-trifluoromethylphenyl]piperidine-1-carboximidamide;
4-fluoro-4-phenyl-N-[4-trifluoromethylphenyl]piperidine-1-carboxamide;
(+/−)-(syn)-4-fluoro-2-methyl-4-(3-methylpyridin-2-yl)-N-[4-trifluoromethylphenyl]piperidine-1-carboxamide;
4-(fluoromethyl)-4-pyridin-2-yl-N-[4-trifluoromethylphenyl]piperidine-1-carboxamide;
syn- and anti-3-fluoro-3-pyridin-2-yl-N-[4-trifluoromethylphenyl]-8-azabicyclo[3.2.1]octane-8-carboxamide & 3-fluoro-3-pyridin-2-yl-N-[4-trifluoromethylphenyl]-8-azabicyclo[3.2.1]octane-8-carboxamide;
4-fluoro-4-pyrimidin-2-yl-N-[4-trifluoromethylphenyl]piperidine-1-carboxamide;
4-fluoro-4-(3-phenylpropyl)-N-[4-trifluoromethylphenyl]piperidine-1-carboxamide;
2-[4-fluoro-4-(3-methylpyridin-2-yl)piperidin-1-yl]-6-trifluoromethyl-1H-benzimidazole;
2-(4-fluoro-4-pyridin-2-ylpiperidin-1-yl)-6-(trifluoromethyl)-1H-benzimidazole;
4-fluoro-N-[4-trifluoromethylphenyl]-4-[3-trifluoromethylpyridin-2-yl]piperidine-1-carboxamide
4-fluoro-N-(4-methylphenyl)-4-(3-methylpyridin-2-yl)piperidine-1-carboxamide;
N-(4-ethylphenyl)-4-fluoro-4-(3-methylpyridin-2-yl)piperidine-1-carboxamide;
N-(4-chlorophenyl)-4-fluoro-4-(3-methylpyridin-2-yl)piperidine-1-carboxamide;
4-fluoro-4-(3-methylpyridin-2-yl)-N-[4-trifluoromethoxyphenyl]piperidine-1-carboxamide;
N-(4-cyanophenyl)-4-fluoro-4-(3-methylpyridin-2-yl)piperidine-1-carboxamide;
N-[4-dimethylaminophenyl]-4-fluoro-4-(3-methylpyridin-2-yl)piperidine-1-carboxamide;
and pharmaceutically acceptable salts thereof.

12. A pharmaceutical composition comprising one or more compounds of claim 10 or 11, or pharmaceutically acceptable salts thereof in association with a pharmaceutically acceptable carrier or excipient.

13. A compound of claim 10 or 11, or a pharmaceutically acceptable salt thereof, for use in treatment of the human or animal body.

14. The use of a compound of claim 10 or 11, or a pharmaceutically acceptable salt thereof for use in the manufacture of a medicament for the treatment or prevention of physiological disorders that may be ameliorated by modulating VR1 activity.

15. The use of a compound of claim 10 or 11, or a pharmaceutically acceptable salt thereof for use in the manufacture of a medicament for the treatment or prevention of a disease or condition in which pain and/or inflammation predominates.

16. The process for the preparation of a compound of claim 10, which comprises:
(A) for compounds wherein Y is NH or NH(CH₂)₁₋₃, reacting a compound of formula (II) with a compound of formula (III): 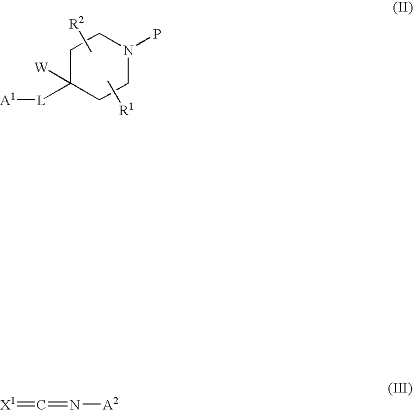
wherein X¹ is O or S, P is H or a C₁₋₆alkoxycarbonyl group such as tert-butoxycarbonyl and A¹, A², L, R¹, R² and W are as defined in claim 10;
(B) for compounds wherein Y is a bond or C₁₋₄alkylene, reacting a compound of formula (II) with a compound of formula (IV): 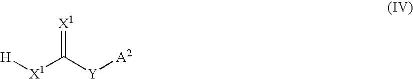
wherein both X¹s are O or S, Y is a bond or C₁₋₄alkylene and A² is as defined in claim 1; or
(C) for compounds wherein X, together with the atom to which it is attached, and Y, form an unsaturated five membered ring together with A², reacting a compound of formula (II) with a compound of formula (V): 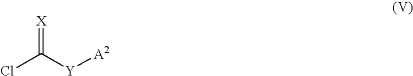
wherein X, together with the atom to which it is attached and Y, form an unsaturated five membered ring together with A².

17. A method for the treatment or prevention of physiological disorders that may be ameliorated by modulating VR1 activity, which method comprises administration to a patient in need thereof of an effective amount of a compound of claim 10 or a composition comprising a compound of claim 10.

18. A method for the treatment or prevention of a disease or condition in which pain and/or inflammation predominates, which method comprises administration to a patient in need thereof of an effective amount of a compound of claim 10, or a composition comprising a compound of claim 10.
